(19) United States
(12) Patent Application Publication
(10) Pub. No.: US 2010/0280509 A1
(43) Pub. Date: Nov. 4, 2010

(54) Eye Therapy System

(75) Inventors: David Muller, Boston, MA (US); Neal Marshall, Ashby, MA (US); Thomas Ryan, Waltham, MA (US)

Correspondence Address: NIXON PEABODY, LLP, 300 S. Riverside Plaza, 16th Floor, CHICAGO, IL, 60606-6613, US

(73) Assignee: Avedro, Inc., Waltham, MA (US)

(21) Appl. No.: 12/753,662

(22) Filed: Apr. 2, 2010

Related U.S. Application Data
(60) Provisional application No. 61/165,998, filed on Apr. 2, 2009.

(51) Int. Cl. A61B 18/18

(52) U.S. Cl. 606/33

(57) ABSTRACT

An electrical energy applicator directs electrical energy from the electrical energy source to a distal end, which positionable at a surface of an eye. The energy conducting applicator includes a first conductor and a second conductor separated by a gap. The first conductor has a first contact surface at the distal end, and the second conductor has a second contact surface at the distal end. The first conductor and/or the second conductor has a length that is adjustable by a biasing element. The first contact surface of the first conductor is movable relative to the second contact surface of the second conductor. The first contact surface and the second contact surface are adjustably positionable simultaneously against the surface of the eye to deliver energy to the eye according to a pattern defined by the first contact surface, the second contact surface, and the gap.

### CROSS-REFERENCE TO RELATED APPLICATIONS

This application claims priority to U.S. Provisional Application No. 61/165,998, filed Apr. 2, 2009, the contents of which are incorporated entirely herein by reference.

### BACKGROUND OF THE INVENTION

1. Field of the Invention

The invention pertains to the field of keratoplasty and, more particularly, to a systems and methods employing an applicator configured to achieve sufficient contact with an eye to apply thermokeratoplasty.

2. Description of Related Art

A variety of eye disorders, such as myopia, keratoconus, and hyperopia, involve abnormal shaping of the cornea. Keratoplasty reshapes the cornea to correct such disorders. For example, with myopia, the shape of the cornea causes the refractive power of an eye to be too great and images to be focused in front of the retina. Flattening aspects of the cornea's shape through keratoplasty decreases the refractive power of an eye with myopia and causes the image to be properly focused at the retina.

Invasive surgical procedures, such as laser-assisted in-situ keratomileusis (LASIK), may be employed to reshape the cornea. However, such surgical procedures typically require a healing period after surgery. Furthermore, such surgical procedures may involve complications, such as dry eye syndrome caused by the severing of corneal nerves.

Thermokeratoplasty, on the other hand, is a noninvasive procedure that may be used to correct the vision of persons who have disorders associated with abnormal shaping of the cornea, such as myopia, keratoconus, and hyperopia. Thermokeratoplasty may be performed by applying electrical energy in the microwave or radio frequency (RF) band. In particular, microwave thermokeratoplasty may employ a near field microwave applicator to apply energy to the cornea and raise the corneal temperature. At about 60° C., the collagen fibers in the cornea shrink. The onset of shrinkage is rapid, and stresses resulting from this shrinkage reshape the corneal surface. Thus, application of heat energy according to particular patterns, including, but not limited to, circular or annular patterns, may cause aspects of the cornea to flatten and improve vision in the eye.

### SUMMARY

In general, the pattern of energy applied to a cornea during thermokeratoplasty depends on the position of the energy applicator relative to the cornea. Thus, to provide reliable application of energy to the cornea, embodiments according to aspects of the present invention position the applicator in uniform and constant contact with the cornea while the applicator provides eye therapy. In this way, the relationship between the applicator and the cornea is more definite and the resulting delivery of energy is more predictable and accurate. The positioning of the applicator provides better electrical and thermal contact. Advantageously, these embodiments also provide a system and method for accurately reproducing sufficient contact between the applicator and the cornea.

An electrical energy applicator in one embodiment extends from a proximal end to a distal end. The energy conducting applicator includes, at the proximal end, a connection to an electrical energy source. The energy conducting applicator directs electrical energy from the electrical energy source to the distal end. The distal end is positionable at a surface of an eye. The energy conducting applicator includes a first conductor and a second conductor separated by a gap. The first conductor has a first contact surface at the distal end, and the second conductor has a second contact surface at the distal end. The first conductor and/or the second conductor has a length that is adjustable by a biasing element. The first contact surface of the first conductor is movable relative to the second contact surface of the second conductor. The first contact surface and the second contact surface are adjustably positionable simultaneously against the surface of the eye to deliver energy to the eye according to a pattern defined by the first contact surface, the second contact surface, and the gap.

In operation, the distal end of the electrical energy applicator is positioned at a surface of an eye, and electrical energy is directed from the electrical energy source to the surface of the eye according to the pattern. For example, the distal end of the electrical energy applicator is positioned by positioning the first contact surface against the eye surface and subsequently moving the second contact surface against the eye surface by compressing the biasing element in the first conductor and reducing the length of the first conductor.

These and other aspects of the present invention will become more apparent from the following detailed description of the preferred embodiments of the present invention when viewed in conjunction with the accompanying drawings.

### BRIEF DESCRIPTION OF THE DRAWINGS

FIG. 1 illustrates a system for applying heat to a cornea of an eye to cause reshaping of the cornea.

FIG. 2A illustrates a high resolution image of a cornea after heat has been applied.

FIG. 2B illustrates another high resolution images of the cornea of FIG. 2A.

FIG. 2C illustrates a histology image of the cornea of FIG. 2A.

FIG. 2D illustrates another histology image of the cornea of FIG. 2A.

FIG. 3A illustrates a view of a system that achieves sufficient contact between the electrical energy conducting element and the eye according to aspects of the present invention.

FIG. 3B illustrates another view of the example configuration of FIG. 3A.

FIG. 3C illustrates example dimensions for a system that achieves sufficient contact between the electrical energy conducting element and the eye according to aspects of the present invention.

### DESCRIPTION

In general, the pattern of energy applied to a cornea during thermokeratoplasty depends on the position of the energy applicator relative to the cornea. Thus, to provide reliable application of energy to the cornea, embodiments according to aspects of the present invention position the applicator in uniform and constant contact with the cornea while the applicator provides eye therapy. In this way, the relationship between the applicator and the cornea is more definite and the resulting delivery of energy is more predictable and accurate. The positioning of the applicator provides better electrical and thermal contact. Advantageously, these embodiments also provide a system and method for accurately reproducing sufficient contact between the applicator and the cornea.

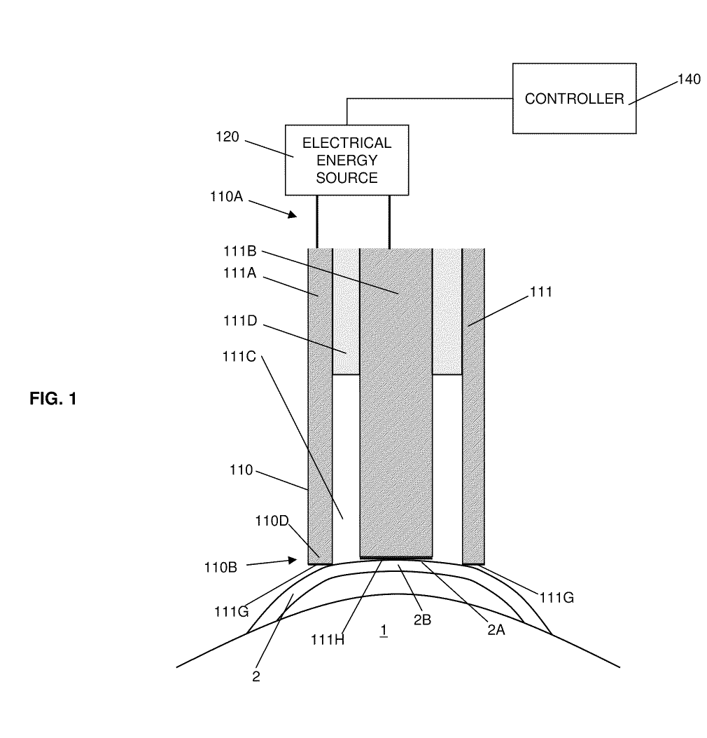

FIG. 1 illustrates an example system for applying energy to a cornea 2 of an eye 1 to generate heat and cause reshaping of the cornea. In particular, FIG. 1 shows an applicator 110 with an electrical energy conducting element 111 that is operably connected to an electrical energy source 120, for example, via conventional conducting cables. The electrical energy conducting element 111 extends from a proximal end 110A to a distal end 110B of the applicator 110. The electrical energy conducting element 111 conducts electrical energy from the source 120 to the distal end 110B to apply heat energy to the cornea 2, which is positioned at the distal end 110B. In particular, the electrical energy source 120 may include a microwave oscillator for generating microwave energy. For example, the oscillator may operate at a microwave frequency range of about 400 MHz to about 3000 MHz, and more specifically at a frequency of about 915 MHz or about 2450 MHz which has been safely used in other applications. As used herein, the term “microwave” may generally correspond to a frequency range from about 10 MHz to about 10 GHz.

As further illustrated in FIG. 1, the electrical energy conducting element 111 may include two microwave conductors 111A and 111B, which extend from the proximal end 110A to the distal end 110B of the applicator 110. In particular, the conductor 111A may be a substantially cylindrical outer conductor, while the conductor 111B may be a substantially cylindrical inner conductor that extends through an inner passage extending through the outer conductor 111A. With the inner passage, the outer conductor 111A has a substantially tubular shape. The outer conductor 111A and the inner conductor 111B may be formed, for example, of aluminum, stainless steel, brass, copper, other metals, coated metals, metal-coated plastic, or any other suitable conductive material.

With the concentric arrangement of conductors 111A and 111B, a substantially annular gap 111C of a selected distance is defined between the conductors 111A and 111B. The annular gap 111C extends from the proximal end 110A to the distal end 110B. A dielectric material 111D may be used in portions of the annular gap 111C to separate the conductors 111A and 111B. The distance of the annular gap 111C between conductors 111A and 111B determines in part the penetration depth of microwave energy into the cornea 2 according to established microwave field theory. Thus, the energy conducting element 111 receives, at the proximal end 110A, the electrical energy generated by the electrical energy source 120, and directs microwave energy to the distal end 111B, where the cornea 2 is positioned.

In general, the outer diameter of the inner conductor 111B may be selected to achieve an appropriate change in corneal shape, i.e. keratometry, induced by the exposure to microwave energy. Meanwhile, the inner diameter of the outer conductor 111A may be selected to achieve a desired gap between the conductors 111A and 111B. For example, the outer diameter of the inner conductor 111B ranges from about 2 mm to about 10 mm while the inner diameter of the outer conductor 111A ranges from about 2.1 mm to about 12 mm. In some systems, the annular gap 111C may be sufficiently small, e.g., in a range of about 0.1 mm to about 2.0 mm, to minimize exposure of the endothelial layer of the cornea (posterior surface) to elevated temperatures during the application of energy by the applicator 110.

A controller 140 may be employed to selectively apply the energy any number of times according to any predetermined or calculated sequence. In addition, the heat may be applied for any length of time. Furthermore, the magnitude of heat being applied may also be varied. Adjusting such parameters for the application of heat determines the extent of changes that are brought about within the cornea 2. Of course, the system attempts to limit the changes in the cornea 2 to an appropriate amount of shrinkage of collagen fibrils in a selected region and according to a selected pattern. When employing microwave energy to generate heat in the cornea 2, for example with the applicator 110, the microwave energy may be applied with low power (of the order of 40 W) and in long pulse lengths (of the order of one second). However, other systems may apply the microwave energy in short pulses. In particular, it may be advantageous to apply the microwave energy with durations that are shorter than the thermal diffusion time in the cornea. For example, the microwave energy may be applied in pulses having a higher power in the range of 500 W to 3 kW and a pulse duration in the range of about 5 milliseconds to about one second.

Referring again to FIG. 1, at least a portion of each of the conductors 111A and 111B may be covered with an electrical insulator to minimize the concentration of electrical current in the area of contact between the corneal surface (epithelium) 2A and the conductors 111A and 111B. In some systems, the conductors 111A and 111B, or at least a portion thereof, may be coated with a material that can function both as an electrical insulator as well as a thermal conductor. A dielectric layer 110D may be employed along the distal end 111B of the applicator 110 to protect the cornea 2 from electrical conduction current that would otherwise flow into the cornea 2 via conductors 111A and 111B. Such current flow may cause unwanted temperature effects in the cornea 2 and interfere with achieving a maximum temperature within the collagen fibrils in a mid-depth region 2B of the cornea 2. Accordingly, the dielectric layer 110D is positioned between the conductors 111A and 111B and the cornea 2. The dielectric layer 110D may be sufficiently thin to minimize interference with microwave emissions and thick enough to prevent superficial deposition of electrical energy by flow of conduction current. For example, the dielectric layer 110D may be a biocompatible material deposited to a thickness of between about 10 and 100 micrometers, preferably about 50 micrometers. As another example, the dielectric layer 110D can be a flexible sheath-like structure of biocompatible material that covers the conductors 111A and 111B at the distal end 110B and extends over a portion of the exterior wall of the outer conductor 111B. As still a further example, the dielectric layer 110D can include a first flexible sheath-like structure of biocompatible material that covers the distal end of the inner conductor 111A and a second flexible sheath-like structure of biocompatible material that covers the distal end of the outer conductor 111B.

In general, an interposing layer, such as the dielectric layer 110D, may be employed between the conductors 111A and 111B and the cornea 2 as long as the interposing layer does not substantially interfere with the strength and penetration of the microwave radiation field in the cornea 2 and does not prevent sufficient penetration of the microwave field and generation of a desired heating pattern in the cornea 2. The dielectric material may be elastic, such as polyurethane and silastic, or nonelastic, such as ceramic of high or low permittivity, Teflon®, and polyimides. The dielectric material may have a fixed dielectric constant or varying dielectric constant by mixing materials or doping the sheet, the variable dielectric being spatially distributed so that it may affect the microwave heating pattern in a customized way. The thermal conductivity of the material may have fixed thermal properties (thermal conductivity or specific heat), or may also vary spatially, through mixing of materials or doping, and thus provide a means to alter the heating pattern in a prescribed manner. Another approach for spatially changing the heating pattern is to make the dielectric sheet material of variable thickness. The thicker region will heat less than the thinner region and provides a further means of spatial distribution of microwave heating.

During operation, the distal end 110B of the applicator 110 as shown in FIG. 1 is positioned on or near the corneal surface 2A. Preferably, the applicator 110 makes direct contact with the corneal surface 2A. In particular, such direct contact positions the conductors 111A and 111B at the corneal surface 2A (or substantially near the corneal surface 2A if there is a thin interposing layer between the conductors 111A and 111B and the corneal surface 2A). Accordingly, direct contact helps ensure that the pattern of microwave heating in the corneal tissue has substantially the same shape and dimension as the gap 111C between the two microwave conductors 111A and 111B.

The system of FIG. 1 is provided for illustrative purposes only, and other systems may be employed to apply heat to cause reshaping of the cornea. Other systems are described, for example, in U.S. patent application Ser. No. 12/208,963, filed Sep. 11, 2008, which is a continuation-in-part application of U.S. patent application Ser. No. 11/898,189, filed on Sep. 10, 2007, the contents of these applications being entirely incorporated herein by reference. As described in U.S. patent application Ser. No. 12/208,963, a cooling system may also be employed in combination with the applicator 110 to apply coolant to the cornea 2 and determine how the energy is applied to the cornea 2.

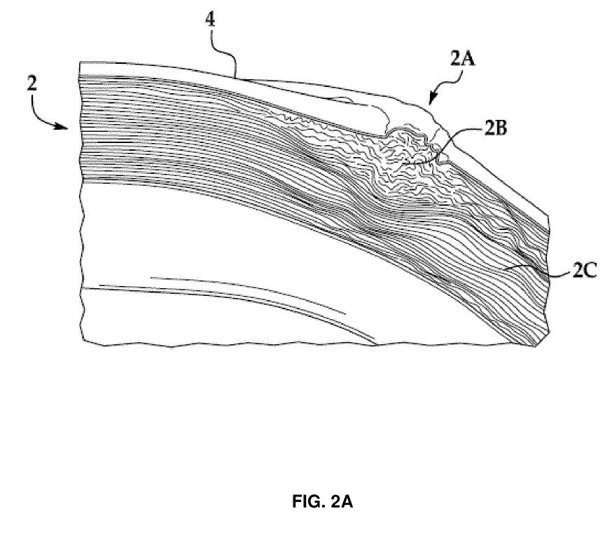

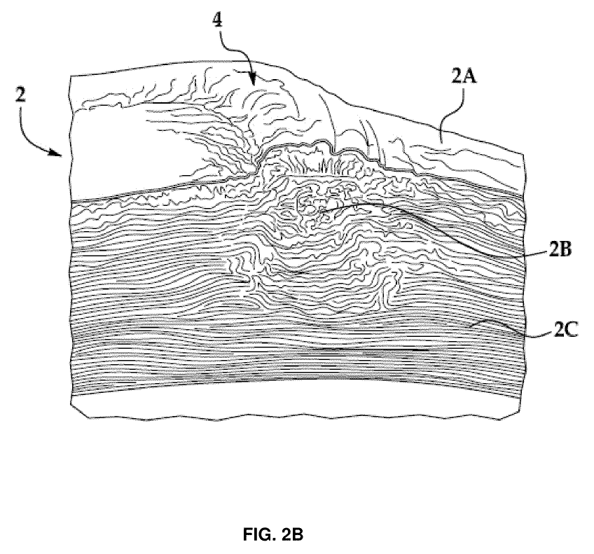

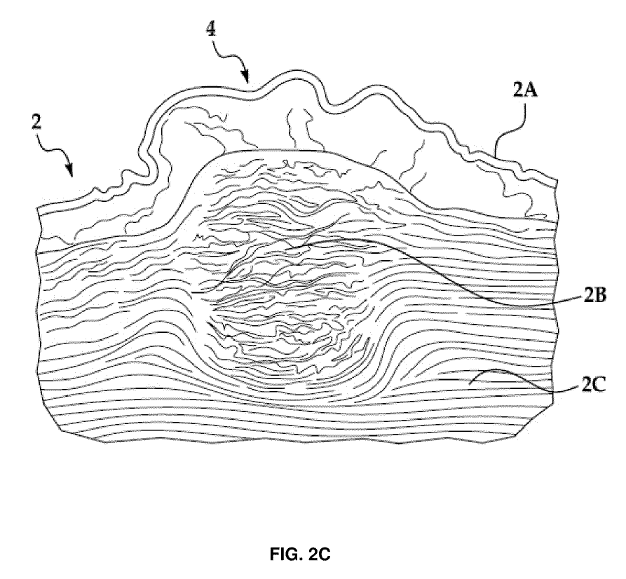

FIGS. 2A-D illustrate an example of the effect of applying heat to corneal tissue with a system for applying heat, such as the system illustrated in FIG. 1. In particular, FIGS. 2A and 2B illustrate high resolution images of cornea 2 after heat has been applied. As FIGS. 2A and 2B show, a lesion 4 extends from the corneal surface 2A to a mid-depth region 2B in the corneal stroma 2C. The lesion 4 is the result of changes in corneal structure induced by the application of heat as described above. These changes in structure result in an overall reshaping of the cornea 2. It is noted that the application of heat, however, has not resulted in any heat-related damage to the corneal tissue.

As further illustrated in FIGS. 2A and 2B, the changes in corneal structure are localized and limited to an area and a depth specifically determined by an applicator as described above. FIGS. 2C and 2D illustrate histology images in which the tissue shown in FIGS. 2A and 2B has been stained to highlight the structural changes induced by the heat. In particular, the difference between the structure of collagen fibrils in the mid-depth region 2B where heat has penetrated and the structure of collagen fibrils outside the region 2B is clearly visible. Thus, the collagen fibrils outside the region 2B remain generally unaffected by the application of heat, while the collagen fibrils inside the region 2B have been rearranged and formed new bonds to create completely different structures. In other words, unlike processes, such as orthokeratology, which compress areas of the cornea to reshape the cornea via mechanical deformation, the collagen fibrils in the region 2B are in an entirely new state.

As shown in FIG. 1, the energy conducting element 111 includes a contact surface 111G at the distal end 110B of the outer conductor 111A and a contact surface 111H at the distal end 110B of the inner conductor 111B. The contact surfaces 111G and 111H come into direct contact with the corneal surface 2A. In general, the application of energy to the cornea 2 depends in part on the position of the contact surfaces 111G and 111H relative to the corneal surface 2A. As a result, to provide reliable application of energy to the cornea 2, embodiments ensure that the contact surfaces 111G and 111H, or portions thereof, are positioned to make sufficient contact with the corneal surface 2A. In this way, the relationship between the energy conducting element 111 and the cornea 2 is more definite and the resulting delivery of energy is more predictable and accurate. Furthermore, safety is enhanced when the applicator 111 is in direct contact with the corneal surface 2A and energy is transferred primarily to the system with good contact. Accordingly, it is preferable not to deliver energy via the energy conducting element 111 unless there is sufficient contact.

In some embodiments, sufficient contact is determined by causing an observable amount of flattening, or applanation, of the cornea. The applanation indicates a constant and uniform pressure against the corneal surface 2A. For example, as illustrated in FIG. 1, the applicator 110 can position the energy conducting element 111 against the corneal surface 2A so that the contact surface 111G flattens the cornea 2. Although the contact surfaces 111G and 111H, or portions thereof, in contact with the corneal surface 2A are shown to be substantially flat in FIG. 1, it is understood that the contact surfaces 111G and 111H may be shaped, e.g., contoured, in other ways to cause the desired contact. The applanation adds precision and accuracy to the eye therapy procedure, particularly by improving electrical and thermal contact between the contact surfaces 111G and 111H and the corneal surface 2A.

Other systems and methods for improving electrical and thermal contact between an energy conducting element and the corneal surface are described in U.S. patent application Ser. No. 12/209,123, filed Sep. 11, 2008, which is a continuation-in-part application of U.S. patent application Ser. No. 12/018,457, filed on Jan. 23, 2008, and U.S. patent Ser. No. 12/617,554, filed on Nov. 12, 2009, which claims priority to U.S. Provisional Patent Application No. 61/113,395, filed Nov. 11, 2008, the contents of these applications being entirely incorporated herein by reference.

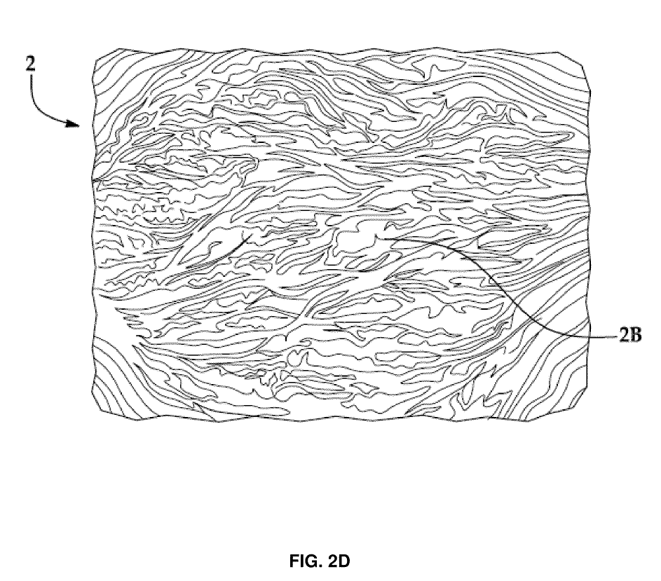

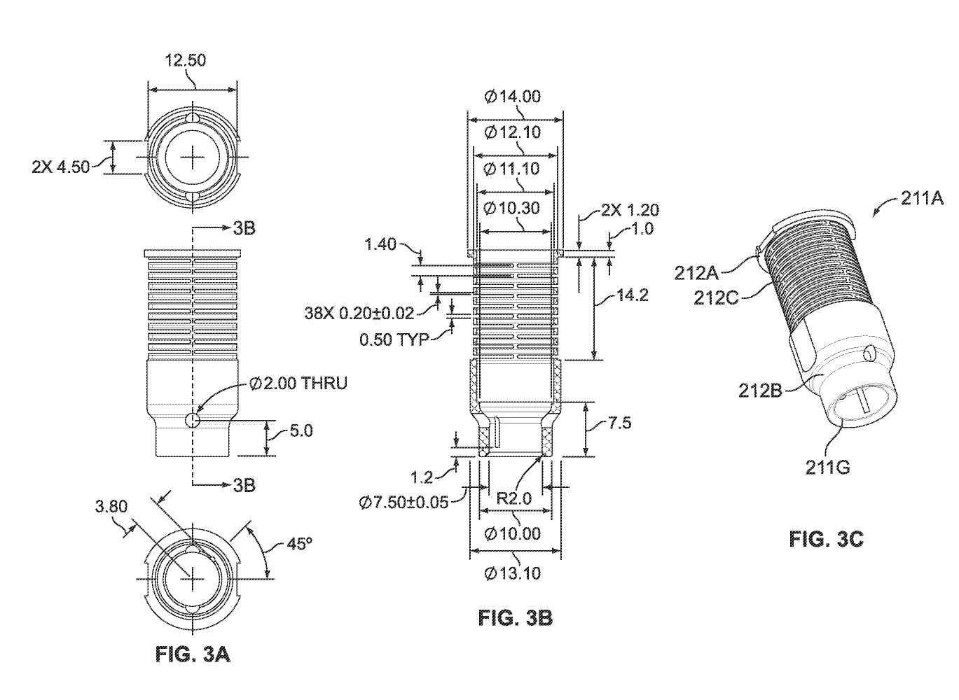

FIGS. 3A-C illustrate an embodiment of an applicator 210 with an energy conducting element 211 that achieves sufficient contact with the cornea 2 of an eye 1. The technique by which the energy conducting element 211 is applied to the cornea 2 may be manual or automated. Like the energy conducting element 111, the energy conducting element 211 includes an outer conductor 211A and an inner conductor 211B that extend along a longitudinal axis 210C from a proximal end 210A to a distal end 210B. The combination of the outer conductor 211A and the inner conductor 211B delivers energy from an energy source 220 to a distal end 210B. The contact surfaces 211G and 211H at the distal end 210B of the outer conductor 211A and the inner conductor 211B, respectively, contact the corneal surface 2A to deliver the energy to the cornea 2. As described previously, the energy is delivered to the cornea 2 in a pattern that depends in part on a gap 211C at the distal end 210B, defined between the outer conductor 211A and the inner conductor 211B. In general, the energy conducting element 211 may be applied to the eye 1 in a manner similar to the energy conducting element 111 to generate heat and cause reshaping of the cornea 2.

Unlike the outer conductor 111A shown in FIG. 1, however, the outer conductor 211A shown in FIGS. 3A-B is configured to provide improved contact between the contact surfaces 211G and 211H and the corneal surface 2A. In particular, the outer conductor 211A includes a proximal section 212A and a distal section 212B connected by a variable section 212C. The proximal section 212A extends from the variable section 212C toward the proximal end 210A where the energy source 220 is connected. The distal section 212B includes the contact surface 211G and extends from the variable section 212C to define the distal end 210B. When assembled, the proximal section 212A, the distal section 212B, and the variable section 212C form a conductive body that allows energy to pass from the proximal section 212A to the distal end 212B via the intermediate device 212C. In addition, the sections 212A, 212B, and 212C each include a central aperture so that they can be aligned along the longitudinal axis 210C to form a passageway through which the inner conductor 211B can extend. Thus, the assembled body in combination with the inner conductor 211B allows energy to be delivered from the proximal end 210A to the distal end 210B as described previously.

The variable section 212C has a length that can vary along the longitudinal axis 210C. For example, the variable section 212C may be adjustably compressed to reduce its length. As the proximal section 212A and the distal section 212B are connected to opposing ends of the variable section 212C, the distal end 212B (and corresponding contact surface 211G) can move relative to the proximal end 212A. This relative movement results in a change in the length of the variable section 212C. Any change in the length of the variable section 212C also corresponds to a change in length of the outer conductor 211A. Thus, when opposing compressive forces are applied against the proximal section 212A and the distal section 212B along the longitudinal axis 210C, the variable section 212C may be compressed and the length of the outer conductor 211A may be reduced.

As shown in FIG. 3A, the applicator 210 may be applied initially to the eye 1 so that at least the contact surface 211G of the outer conductor 211A contacts the corneal surface 2A. As further illustrated in FIG. 3A, however, the inner electrode 211B may be recessed within the inner passage of the outer conductor 211A, so that the contact surface 211G of the outer conductor 211A may achieve sufficient contact with the corneal surface 2A before the corresponding contact surface 211H of the inner electrode 211B achieves sufficient contact with the corneal surface 2A. As described previously, without sufficient contact between the contact surfaces 211G and 211H and the corneal surface 2A, the desired delivery of energy to the cornea 2 may not be possible.

However, as also described previously, the variable section 212C allows the distal section 212B to move relative to the proximal section 212A. In fact, the variable section 212C generally allows the distal section 212B to move relative to the rest of the energy conducting element 211, including the inner conductor 211B. As a result, the configuration of the energy conducting element 211 is not fixed and can be changed to allow both the inner conductor 211B and the outer conductor 211A to achieve sufficient contact with the cornea 2. In effect, the degree to which the inner conductor 211B is recessed within the outer conductor 211A is adjustable to achieve the appropriate geometry for the energy conducting electrode 211.

As FIG. 3B illustrates, the energy conducting element 211 may be moved further in the direction A into contact with the cornea 2. With other energy conducting elements, this movement may increase the pressure applied by the outer conductor 211A to unacceptable levels or damage the cornea 2 before sufficient contact between the inner conductor 211B and the cornea are achieved. In the embodiment of FIG. 3B, however, the cornea 2 applies a reaction force in the direction B against the contact surface 211G of the outer conductor 211A, and this reaction force pushes against the distal section 212B and causes the variable section 212C to compress. As such, the distal section 212B also moves in the direction B. Because an opposing compressive force is applied to the proximal section 212A as the energy conducting element 211 is moved or held against the cornea 2, the distal section 212B moves relative to the proximal section 212A. Moreover, the inner conductor 211B may be generally fixed with respect to the proximal section 212A, so that the distal section 212B also moves relative to the inner conductor 211B. Thus, although the contact surface 211H of inner conductor 211B may continue to move in the direction A against the cornea 2, FIG. 3B shows that relative movement by the contact surface 211G of the outer conductor 211A in the direction B ensures that the pressure between the contact surface 211G does not become excessive.

Furthermore, even though the distal section 212B may move relative to the inner conductor 211B, the desired contact between the contact surface 211G and the cornea 2 is maintained, so that both contact surfaces 211G and 211H achieve sufficient contact once the inner conductor 211B is moved the necessary distance against the cornea 2. In particular, the variable section 212C may provide a bias against a change in length, so that contact between the cornea 2 and the contact surface 211G must be maintained to provide the necessary force against the distal section 212B to keep the variable section 212C compressed. For example, as shown in FIGS. 3A-C, the variable section 212C may be a coil spring, or similar biasing device, that has a spring constant (k) and provides a reaction force (F=−kx) according to a change in length (x) of the spring. The spring constant (k) may be chosen to ensure that there is sufficient bias to maintain contact without requiring too much force to compress the spring. Accordingly, as the energy conducting element 211 is applied to move the inner conductor 211B against the cornea 2, the outer electrode 211A is simultaneously compressed against the cornea 2 to maintain sufficient contact between the contact surface 211G and the corneal surface 2A.

In some embodiments, a sensor system may be coupled to the outer conductor 211A and/or the inner conductor 211B to monitor the force being applied against the eye. The signal from the sensor system may indicate that the desired contact has been achieved or may provide an alert when excessive contact force is applied to the eye.

In other embodiments, the amount of contact between the energy conducting element 211 and the eye may be determined by measuring the effect of sending low level pulses of microwaves from the energy source through the energy conducting element 211. These low level pulses, also known as “sounding pulses,” have a lower power than pulses employed for treatment. When the outer conductor 211A and the inner conductor 211B are only in contact with air at the distal end 210B and are not in contact with an eye, the electrical impedance is generally very high. This impedance may be calculated by sending sounding pulses through the outer conductor 211A and the inner conductor 211B. The sounding pulses also cause power to be reflected within the energy conducting element 211, and this reflected power has a higher value when the outer conductor 211A and the inner conductor 211B are not in contact with tissue. As the energy conducting element 211 comes into contact with tissue, the impedance changes and the reflected power decreases. Thus, the change in contact between the energy conducting electrode 211 and the eye may be dynamically monitored by measuring changes in the impedance or reflected power. An example of a system that monitors contact by measuring reflected power in an energy conducting electrode is described in U.S. patent Ser. No. 12/617,554, filed on Nov. 12, 2009, which claims priority to U.S. Provisional Patent Application No. 61/113,395, filed on Nov. 11, 2008, the contents of these applications being entirely incorporated herein by reference.

FIG. 3C provides an example shape and example dimensions for an outer conductor 211A that is configured with a spring 212C. The outer conductor 211A may be formed from aluminum alloy 7075, for example.

In sum, the FIGS. 3A-3C illustrate an outer conductor 211A that has a section 212C that allows the contact surface 211G of the outer conductor 211A to move relative to the contact surface 211H of the inner conductor 211B. This relative movement allows both the outer conductor 211A and the inner conductor 211B to accommodate the aspects of the eye and achieve sufficient contact for the desired delivery of energy to the cornea 2. As shown in FIG. 3B, the application of the energy conducting element 211 may cause applanation of the cornea 2, providing a visible indication of the contact that is achieved therebetween.

In general, however, the embodiment of FIGS. 3A-3C demonstrates how a variable component, such as a spring, may be employed to provide an energy conducting electrode with an adjustable configuration. As such, the use of the variable component is not limited to the outer conductor. For example, a spring may additionally or alternatively be employed with the inner conductor. In these other embodiments, the contact surfaces of the outer conductor and the inner conductor are also able to move relative to each other.

Although the embodiments described herein may apply energy to the cornea according to an annular pattern defined by an applicator such as the applicators 110 and 210, the pattern in other embodiments is not limited to a particular shape. Indeed, energy may be applied to the cornea in non-annular patterns. Examples of the non-annular shapes by which energy may be applied to the cornea are described in U.S. patent Ser. No. 12/113,672, filed on May 1, 2008, the contents of which are entirely incorporated herein by reference.

Furthermore, the controller 140 described above may be a programmable processing device that executes software, or stored instructions, and that may be operably connected to the other devices described above. In general, physical processors and/or machines employed by embodiments of the present invention for any processing or evaluation may include one or more networked or non-networked general purpose computer systems, microprocessors, field programmable gate arrays (FPGAs), digital signal processors (DSPs), micro-controllers, and the like, programmed according to the teachings of the exemplary embodiments of the present invention, as is appreciated by those skilled in the computer and software arts. The physical processors and/or machines may be externally networked with the image capture device, or may be integrated to reside within the image capture device. Appropriate software can be readily prepared by programmers of ordinary skill based on the teachings of the exemplary embodiments, as is appreciated by those skilled in the software art. In addition, the devices and subsystems of the exemplary embodiments can be implemented by the preparation of application-specific integrated circuits (ASICs) or by interconnecting an appropriate network of conventional component circuits, as is appreciated by those skilled in the electrical art(s). Thus, the exemplary embodiments are not limited to any specific combination of hardware circuitry and/or software.

Stored on any one or on a combination of computer readable media, the exemplary embodiments of the present invention may include software for controlling the devices and subsystems of the exemplary embodiments, for driving the devices and subsystems of the exemplary embodiments, for enabling the devices and subsystems of the exemplary embodiments to interact with a human user, and the like. Such software can include, but is not limited to, device drivers, firmware, operating systems, development tools, applications software, and the like. Such computer readable media further can include the computer program product of an embodiment of the present inventions for performing all or a portion (if processing is distributed) of the processing performed in implementing the inventions. Computer code devices of the exemplary embodiments of the present inventions can include any suitable interpretable or executable code mechanism, including but not limited to scripts, interpretable programs, dynamic link libraries (DLLs), Java classes and applets, complete executable programs, and the like. Moreover, parts of the processing of the exemplary embodiments of the present inventions can be distributed for better performance, reliability, cost, and the like.

Common forms of computer-readable media may include, for example, a floppy disk, a flexible disk, hard disk, magnetic tape, any other suitable magnetic medium, a CD-ROM, CDRW, DVD, any other suitable optical medium, punch cards, paper tape, optical mark sheets, any other suitable physical medium with patterns of holes or other optically recognizable indicia, a RAM, a PROM, an EPROM, a FLASH-EPROM, any other suitable memory chip or cartridge, a carrier wave or any other suitable medium from which a computer can read.

Although the application of the embodiments described herein may be described with respect to the cornea, it is understood that aspects of the present invention may be applied to other features of the eye or anatomy.

While the present invention has been described in connection with a number of exemplary embodiments, and implementations, the present inventions are not so limited, but rather cover various modifications, and equivalent arrangements.

What we claim is:

1. A device for applying therapy to an eye, the system comprising:
an electrical energy applicator extending from a proximal end to a distal end, the energy conducting applicator including, at the proximal end, a connection to an electrical energy source, the energy conducting applicator being adapted to direct electrical energy from the electrical energy source to the distal end, the distal end being positionable at a surface of an eye, the energy conducting applicator including a first conductor and a second conductor separated by a gap, the first conductor having a first contact surface at the distal end, the second conductor having a second contact surface at the distal end, at least one of the first conductor and the second conductor having a length that is adjustable by a biasing element, the first contact surface of the first conductor being movable relative to the second contact surface of the second conductor, the first contact surface and the second contact surface being adjustably positionable simultaneously against the surface of the eye to deliver energy to the eye according to a pattern defined by the first contact surface, the second contact surface, and the gap.

2. The device of claim 1, wherein the first conductor is an outer conductor and the second conductor is an inner conductor disposed in the first conductor.

3. The device of claim 2, wherein outer conductor and the inner conductor are substantially cylindrical and concentric at the distal end, and the gap at the distal end is substantially annular.

4. The device of claim 2, wherein the biasing element forms a part of a conducting body of the outer conductor, the second contact surface of the inner conductor is recessed relative to the first contact surface of the outer conductor, and the length of the outer conductor is reduced by compressing the biasing element when the first contact surface of the outer conductor is positioned against the eye surface to allow the second contact surface of the inner conductor to be moved against the eye surface.

5. The device of claim 1, wherein the gap is non-annular or asymmetric.

6. The device of claim 1, wherein at least one of the first conductor and the second conductor includes a distal segment at the distal end, the distal segment being connected to the biasing element and being movable relative to the proximal end.

7. The device of claim 1, wherein the biasing element forms a part of a conducting body of the first conductor or the second conductor.

8. The device of claim 1, wherein the biasing device is a coil spring.

9. The device of claim 1, wherein one of the first contact surface and the second contact surface is recessed relative to the other of the first contact surface and the second surface.

10. The device of claim 1, wherein the energy conducting applicator includes a sensor system that senses contact between at least one of the first contact surface and the second contact surface against the surface of an eye.

11. A method for applying therapy to an eye, the method comprising:
positioning a distal end of an electrical energy applicator at a surface of an eye, the electrical energy applicator extending from a proximal end to the distal end, the energy conducting applicator including, at the proximal end, a connection to an electrical energy source, the energy conducting applicator including a first conductor and a second conductor separated by a gap, the first conductor having a first contact surface at the distal end, the second conductor having a second contact surface at the distal end, at least one of the first conductor and the second conductor having a length that is adjustable by a biasing element, the first contact surface of the first conductor being movable relative to the second contact surface of the second conductor, the first contact surface and the second contact surface being adjustably positionable simultaneously against the surface of the eye; and
directing, via the energy conducting applicator, electrical energy from the electrical energy source to the surface of the eye according to a pattern defined by the first contact surface, the second contact surface, and the gap.

12. The method of claim 11, wherein positioning the distal end of the electrical energy applicator at the surface of the eye comprises:
positioning the first contact surface against the eye surface; and
moving the second contact surface against the eye surface by compressing the biasing element in the first conductor and reducing the length of the first conductor.

13. The method of claim 11, wherein the first conductor is an outer conductor and the second conductor is an inner conductor disposed in the first conductor.

14. The method of claim 13, wherein outer conductor and the inner conductor are substantially cylindrical and concentric at the distal end, and the gap at the distal end is substantially annular.

15. The method of claim 13, wherein the biasing element forms a part of a conducting body of the outer conductor, the second contact surface of the inner conductor is recessed relative to the first contact surface of the outer conductor, and positioning the distal end of the electrical energy applicator at the surface of the eye comprises:
positioning the first contact surface of the outer conductor against the eye surface; and
moving the second contact surface of the inner conductor against the eye surface by compressing the biasing element in the outer conductor and reducing the length of the outer conductor.

16. The method of claim 11, wherein the gap is non-annular or asymmetric.

17. The method of claim 11, wherein at least one of the first conductor and the second conductor includes a distal segment at the distal end, the distal segment being connected to the biasing element and being movable relative to the proximal end.

18. The device of claim 1, wherein the biasing element forms a part of a conducting body of the first conductor or the second conductor.

19. The method of claim 11, wherein one of the first contact surface and the second contact surface is recessed relative to the other of the first contact surface and the second surface.

20. The method of claim 11, further comprising sensing contact between at least one of the first contact surface and the second contact surface against the surface of an eye.
